(19) United States
(12) Patent Application Publication
(10) Pub. No.: US 2025/0246307 A1
(43) Pub. Date: Jul. 31, 2025

(54) PREDICTION METHOD AND PREDICTION SYSTEM FOR MRKH SYNDROM, AND DEVICE

(71) Applicant: PEKING UNION MEDICAL COLLEGE HOSPITAL, CHINESE ACAEMY OF MEDICAL SCIENCES, Beijing (CN)

(72) Inventors: Lan ZHU, Beijing (CN); Na CHEN, Beijing (CN); Nan WU, Beijing (CN); Sen ZHAO, Beijing (CN); Xi CHENG, Beijing (CN)

(21) Appl. No.: 19/040,239

(22) Filed: Jan. 29, 2025

(30) Foreign Application Priority Data
Jan. 31, 2024 (CN) 202410133356.6

(51) Int. Cl. G16H 50/20; G16B 20/20; G16B 25/10; G16B 40/30; G16H 10/40; G16H 10/60; G16H 50/30; G16H 50/70

(52) U.S. Cl. CPC G16H 50/20; G16B 20/20; G16B 25/10; G16B 40/30; G16H 10/40; G16H 10/60; G16H 50/30; G16H 50/70

(57) ABSTRACT

Provided are a prediction method and a prediction system for MRKH (Mayer-Rokitansky-Küster-Hauser) syndrome, a device, a medium, and a program product, relating to the field of intelligent healthcare. The method includes the following steps: acquiring gene expression data of a sample to be tested; extracting expression data of target genes from the gene expression data, where the target genes include one or more of the following: AGPAT2, and PAN2; performing classification prediction based on the expression data of the target genes to obtain a classification result about whether the sample to be tested suffers from MRKH; if expression level of any one or more of the target genes is high, obtaining a classification result that the sample to be tested suffers from MRKH; and on the contrary, obtaining a classification result that the sample to be tested does not suffer from MRKH.

### CROSS-REFERENCE TO RELATED APPLICATION

This patent application claims the benefit and priority of Chinese Patent Application No. 202410133356.6 entitled “Prediction Method and Prediction System for MRKH Syndrome, and Device” filed with the China National Intellectual Property Administration on Jan. 31, 2024, the disclosure of which is incorporated by reference herein in its entirety as part of the present application.

### TECHNICAL FIELD

The present disclosure relates to the field of intelligent healthcare, more specifically a prediction method and a prediction system for MRKH syndrome, a device, a medium, and a program product.

### BACKGROUND

MRKH (Mayer-Rokitansky-Küster-Hauser) syndrome, also known as congenital absence of vagina syndrome, is a congenital reproductive system structural defect of female. The clinical characteristics and manifestations of the patient are complete congenital absence of uterus and upper 2/3 segments of vagina (even primordial uterus), with normal ovarian reproductive and endocrine functions, normal development of secondary sexual characteristics, chromosome karyotype of 46, XX, and an incidence rate of about 1/4500-5000 newborn baby girls. MRKH syndrome can be divided into two types according to whether complicated with malformations of other systems outside the reproductive tract. Type I MRKH syndrome is simple reproductive tract malformation accounting for 44% of patients with MRKH syndrome, and Type II MRKH syndrome is a MRKH syndrome that is complicated with renal and/or skeletal system dysplasia accounting for 56% of patients with MRKH syndrome. The most common malformations observed in 40%-60% patients with Type II MRKH syndrome are unilateral renal absence and/or renal ectopia.

Although there is no vertical transmission, family aggregation rarely occurs. However, Mayer-Rokitansky-Küster-Hauser syndrome (MRKHS) has a significant genetic susceptibility. WNT4 has been identified as a pathogenic gene of MRKHS for the first time through case analysis. Repeated copy number variation (CNV), such as absence of 16p11.2 and duplication of 17q12, may also lead to incomplete penetrance pathogenesis of MRKHS. Through parallel sequencing of case-control cohort, MRKHS related genes were identified in PAX8, TBX6, LHX1 and BMP7 by candidate gene strategies. The collection of candidate genes in previous studies was mainly based on the developmental biology of uterus. PAX8 and LHX1 are responsible for regulation of the specifications of mesodermal epithelial progenitor cells and the invagination of Müllerian Duct (MD) (primitive primordia of female reproductive tract). WNT4, as a signal molecule of the mesenchymal precursor, is particularly important for the prolongation of MD. In addition, coelom epithelium (CE), Wolffian Duct (WD) and various other cell lineages are also crucial for the development of uterus. Therefore, deciphering the molecular networks of different cell types involved in uterine development may promote the prioritization of new MRKHS-related genes. Conversely, the disease genes identified by phylogenetic analysis can tell the key developmental stages and cell types of human body. For example, the expression of genes related to amyotrophic lateral sclerosis (ALS) is enriched in glutamatergic neurons of all cell types in the brain, which indicates that neuron-specific pathology of ALS can accurately locate potential drug targets. However, there is no single-cell transcriptome of embryonic uterus, and there is no systematic genetic association study on MRKHS at present.

### SUMMARY

The present disclosure aims at solving at least one of the technical problems existing in the prior art. To this end, the present disclosure provides a prediction method and system for MRKH syndrome. By detecting an expression level of a target gene of a sample to be tested, a classification result about whether the sample to be tested suffers from MRKH is predicted, thus providing information for prenatal care and birth defect prevention; complex genetic factors related to MRKHS are revealed, and biological relevance thereof in the context of single-cell transcriptome of the embryonic uterus is described through comprehensive analysis of genome and embryonic transcriptome data, thus solving related life science problems.

A first aspect of the present disclosure provides a prediction method for MRKH syndrome, including the following steps:
acquiring gene expression data of a sample to be tested;
extracting expression data of target genes from the gene expression data, where the target genes include one or more of the following: AGPAT2, and PAN2; and
performing classification prediction based on the expression data of the target genes to obtain a classification result about whether the sample to be tested suffers from MRKH; if expression level of any one or more of the target genes is high, obtaining a classification result that the sample to be detected suffers from MRKH; and if the expression level of any one or more of the target genes is low, obtaining a classification result that the sample to be tested does not suffer from MRKH.

In some embodiments, the target gene further includes one or more of the following: PAX8, BMP7, and GREB1L;

Further, the target gene further includes any one or more of the following suboptimal genes: MT1F, BMP7, and AGPAT2.

Further, the target gene further includes one or more of the following digenic combinations: EVC2-KANK1, CYP2A7-CPSF3L, NOS1-AICDA, AGPAT2-MAMDC4, BMP7-SH2D6, and PAX8-MYO7A.

In some embodiments, the method further includes the following steps: acquiring a genome mutation burden score of the sample to be tested, and obtaining the classification result for predicting the classification result of high or low risk of suffering from MRKH according to the score; if the genome mutation burden score is high, obtaining a classification result that the sample to be tested has a high risk of suffering from MRKH; and if the genome mutation burden score is low, obtaining a classification result that the sample to be tested has low risk of suffering from MRKH.

Further, the genome mutation burden score is obtained through the following ways: screening out rare variants in the target genes by using a mutation effect prediction tool, endowing weight values respectively according to the harmful degree of the rare variants to gene function, and comprehensively weighting to calculate the genome mutation burden score of the sample to be tested.

Alternatively, the genome mutation burden score is further obtained through the following ways: screening out a rare variant in each gene by using the mutation effect prediction tool, endowing weight values respectively according to the harmful degree of each rare variant to the gene function, and comprehensively weighting to calculate the genome mutation burden score of the sample to be tested.

In some embodiments, the expression levels of any one or more of the target genes of the sample to be tested at an 8th week and a 11th week are acquired, if the expression levels of the target genes are high in uterine epithelial cells at the 8th week and Wolffian Duct epithelial cells at the 11th week, a result that the sample to be tested suffers from MRKH is obtained. The sample to be tested is a developing embryo.

In some embodiments, the classification result is obtained based on a prediction model, and a construction method for the prediction model includes the following steps:
acquiring genome sequencing data of a sample from a training set and corresponding clinical characteristics of the sample, where the clinical characteristics are collected from MRKH patients and healthy people;
decomposing an ultra-rare variant in the genome sequencing data into gene units by using a weighting system, and performing burden testing on the MRKH patient and healthy people to obtain a target gene with significant expression in whole exome and an expression level of the target gene, alternatively, the weighting system is a six-layer weighting system; and
modeling based on the expression level of the target gene as well as the clinical characteristics to obtain a prediction model.

In some embodiments, the construction method for the prediction model further includes the following steps:
acquiring gene expression data of significant genes in metanephric cells of human and mouse at different embryonic stages;
extracting gene expression data of the target genes with significant expression after removing a whole exome signal of the target gene from the gene expression data of the target genes with significant expression to obtain a first suboptimal gene related to MRKH, where the first suboptimal gene includes: MT1F; and
modeling based on expression levels of the target gene and the first suboptimal gene as well as the clinical characteristics to obtain a prediction model.

In some embodiments, the construction method for the prediction model further includes the following steps: extracting MRKH-related digenic combinations from the genome sequencing data, and screening from the MRKH-related digenic combinations to obtain a first digenic combination, where the first digenic combination includes EVC2-KANK1, CYP2A7-CPSF3L, NOS1-AICDA; and a screening criterion of the first digenic combination includes: identifying a combination of rare variants in MRKH-related digenic combinations by RareComb tool, and including the mutation predicted as harmful mutation in the weight system into the analysis. A total of 992 digenic combinations are identified, three of which are significant by a corrected P value.

A second digenic combination is determined from the digenic combinations including the target gene. The second digenic combination includes: AGPAT2-MAMDC4, BMP7-SH2D6, and PAX8-MYO7A. A screening criterion of the second digenic combination includes: screening all digenic combinations including the target genes with significant expression in whole exome, and identifying three (significant) digenic combinations with P values of less than 0.05.

Modeling based on expression levels of the target gene and the digenic combination as well as the clinical characteristics to obtain a prediction model.

In some embodiments, the construction method for the prediction model further includes the following steps:
acquiring transcriptome sequencing data of a sample from a training set, and performing hierarchical clustering on gene expression patterns of the transcriptome sequencing data to obtain at least one co-expressed gene cluster; screening from the co-expressed gene cluster to obtain a gene that does not reach the significance of the whole exome but is clustered into a same gene module as the whole exome signal, which is recorded as a second suboptimal gene; where the second suboptimal gene comprises BMP7, and AGPAT2;
decomposing an ultra-rare variant in the co-expressed gene cluster into genes by using a weighting system, summing a maximum weight burden score of each decomposed gene to calculate a genome mutation burden score, and fitting the genome mutation burden scores to obtain a fitting result; and
modeling based on expression levels of the target genes and the suboptimal gene, as well as the fitting result and the clinical characteristics to obtain a prediction model;
a second aspect of the present disclosure provides a prediction system for a MRKH syndrome.

The system includes:
an acquisition unit, configured to acquire gene expression data of a sample to be tested;
an extraction unit, configured to extract expression data of a target gene from the gene expression data, where the target gene includes one or more of the following: AGPAT2, and PAN2; and
a prediction unit, configured to perform classification prediction based on the expression data of the target gene to obtain a classification result about whether the sample to be tested suffers from MRKH, obtain a classification result that the sample to be detected suffers from MRKH if expression level of any one or more of the target genes is high, and obtain a classification result that the sample to be tested does not suffer from MRKH if the expression level of any one or more of the target genes is low.

A third aspect of the present disclosure provides a computer device, including a memory and a processor. A program instruction is stored in the memory, and a program instruction is called in the processor, and the program instruction, when being executed, is configured to execute the steps of the method above.

A fourth aspect of the present disclosure discloses a computer readable storage medium, a computer program is stored thereon. The computer program, when being executed by a processor, is configured to implement the steps of the method above.

A fifth aspect of the present disclosure provides a computer program product including a computer program. The computer program, when being executed by a processor, is configured to implement the steps of the method above.

The present disclosure has the following beneficial effects:

1. The present disclosure innovatively discloses a prediction method for MRKH syndrome. By the method, biological relevance of genome and embryo in the context of single-cell transcriptome of the embryonic uterus is described through comprehensive analysis of genome and embryonic transcriptome data, and the classification result of whether the sample to be tested has MRKH or not is achieved by prediction by detecting the expression level of the target gene of the sample to be tested at the stage of practical application, so as to provide information for prenatal care and birth defects prevention;

2. Because MRKHS is rare and not by vertical transmission, it is difficult to find a gene involved in the disease. In this application, gene-based association strategies are innovatively used to study the contribution made by ultra-rare variants to MRKHS. Specifically, the known MRKHS-related genes are “rediscovered”, including PAX8, BMP7 and GREB1L, thus verifying the relationship between the genes and disease in an unknowable way. Two genes (AGPAT2 and PAN2) with whole exome significance are also identified to be involved in the disease, and their expression levels in developing uterus are tested by IHC (immunohistochemistry) and scRNA-seq. Both AGPAT2 and PAN2 are associated with autosomal recessive inheritance syndrome, and have phenotypic spectrum involving urogenital malformation. In these cases, only single allelic variants of AGPAT2 and 6PAN2 are found, which corresponds to an increased risk of disease, not Mendelian inheritance.

3. In this application, the method of “forward genetics” is used to study human embryogenesis, and the key embryonic stages and key cell types involved in the pathogenesis of MRKHS are determined through the enrichment analysis of the expression of the MRKHS-related genes. It is found that WD epithelium is enriched due to the expression of a burden test signal, which reiterates the important role of MD-WD interaction in uterine development. The dependence of the MD development on WD is also revealed in a mutant mouse model, in which WD fails to form, degenerates shortly after formation, or lacks key signal molecules. In addition to expression analysis, the mutation burden of cell-type-specific-driver genes is also tested based on a cell velocity model. In addition to the expression spectra of different developmental stages, the model also provides a dynamic view. Knowledge about when and which part of the developing uterus is the most vulnerable can provide information for prenatal care and prevention of birth defects.

In a word, in this study, the complex genetic factors related to MRKHS are revealed, and their biological relevance in the context of single-cell transcriptome of the embryonic uterus is described.

### BRIEF DESCRIPTION OF THE DRAWINGS

To describe the technical solutions of the embodiments of the present disclosure more clearly, the following briefly introduces the accompanying drawings required for describing the embodiments. Apparently, the accompanying drawings in the following description show merely some embodiments of the present disclosure, and those of ordinary skill in the art may still derive other drawings from these accompanying drawings without creative efforts.

FIG. 1 is a flow diagram of a method according to a first aspect of an embodiment of the present disclosure;

FIG. 2 is a flow diagram of a system according to a second aspect of an embodiment of the present disclosure;

FIG. 3 is a schematic diagram of a computer device according to an embodiment of the present disclosure.

FIGS. 4A-4B are schematic expression diagrams of a gene-based mutation burden test according to an embodiment of the present disclosure (where FIG. 4A is a quantile-quantile diagram of weighted burden analysis and FIG. 4B is a diagram of synonymous variant burden analysis; genes with whole exome significance are marked in red; and the Synonymous burden test shows that a pipeline is well calibrated);

FIG. 5 is a schematic expression diagram of some top genes according to an embodiment of the present disclosure (the expression of new disease genes (PAN2 and AGPAT2) and two known disease genes (PAX8 and BMP7) in the metanephric single-cell transcriptome of human and mouse);

FIG. 6 is a schematic distribution diagram of some ultra-rare variants in PAN2 according to an embodiment of the present disclosure, where USP indicates an ubiquitin-specific protease, and WD, WD40 are repeat domains;

FIG. 7 is a schematic distribution diagram of an ultra-rare variant in AGPAT2 according to an embodiment of the present disclosure;

FIGS. 8A-8B are schematic diagrams of immunohistochemical staining of PAN2 and AGPAT2 proteins using E13.5 mouse metanephros according to an embodiment of the present disclosure;

FIG. 9 is a schematic diagram of enrichment of MRKHS related genes in different development stages of metanephric single-cell transcriptome of human according to an embodiment of the present disclosure;

FIG. 10 is a schematic diagram of enrichment of MRKHS related genes in different development stages of metanephric single-cell transcriptome of mouse according to an embodiment of the present disclosure;

FIG. 11 is a schematic diagram of co-expression analysis of a gene module derived from metanephric single-cell transcriptome of human according to an embodiment of the present disclosure;

FIG. 12 is a schematic diagram of enrichment of MRKHS related genes in different development stages of metanephric single-cell transcriptome of mouse according to an embodiment of the present disclosure;

FIG. 13 is a Manhattan diagram showing a genome level burden test of biological pathway according to an embodiment of the present disclosure, in which the full path significance (red line) is defined as a false discovery rate (FDR) of less than 0.05. A database for searching the biological pathway includes gene ontology biological process (GOBP), a pathway interaction database (PID), Wiki Pathway (WP), Kyoto encyclopedia of genes and genomes (KEGG), Reactome and Biocarta Pathways data sets;

FIG. 14 is a schematic illustration diagram of a role of mesenchymal-epithelial transformation and apoptosis in uterine development according to an embodiment of the present disclosure;

FIG. 15 is a schematic diagram of a gene set level burden test of biological pathway and cell-type-specific-driver gene in metanephric single-cell transcriptome of human according to an embodiment of the present disclosure;

FIG. 16 is a schematic diagram of a gene set level burden test of biological pathway and cell-type-specific-driver gene in metanephric single-cell transcriptome of mouse according to an embodiment of the present disclosure;

FIG. 17 is a chromosome-based circos diagram of some digenic combinations with significance in whole exome according to an embodiment of the present disclosure.

FIG. 18 is a schematic diagram showing cell-type-specific co-expression score between KANK1 and EVC2 in metanephric single-cell transcriptome of human (left) and mouse (right) according to an embodiment of the present disclosure;

FIG. 19 is a schematic diagram of specific flow of a six-layer weighting system according to an embodiment of the present disclosure.

### DETAILED DESCRIPTION OF THE EMBODIMENTS

In order to make those skilled in the art understand the solutions of the present disclosure better, the technical solutions in the embodiments of the present disclosure will be described clearly and completely in conjunction with the accompanying drawings.

In some processes described in the specification and claims of the present disclosure and the above accompanying drawings, multiple operations appear in a specific order. However, it should be clearly understood that these operations may be executed in an order other than that appeared herein or may be performed in parallel. Sequence numbers of the operations (e.g., 101, 102, etc.) are only used to distinguish different operations, and the sequence numbers themselves do not represent any required execution order. In addition, these processes may include more or fewer operations, and these operations may be performed in sequence or in parallel. It should be noted that the descriptions such as “first” and “second” herein are used to distinguish different messages, devices, modules, etc., and do not represent a sequential order, nor limit “first” and “second” as different types.

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by those of ordinary skill in the art based on the embodiments of the present disclosure without creative efforts shall fall within the protection scope of the present disclosure.

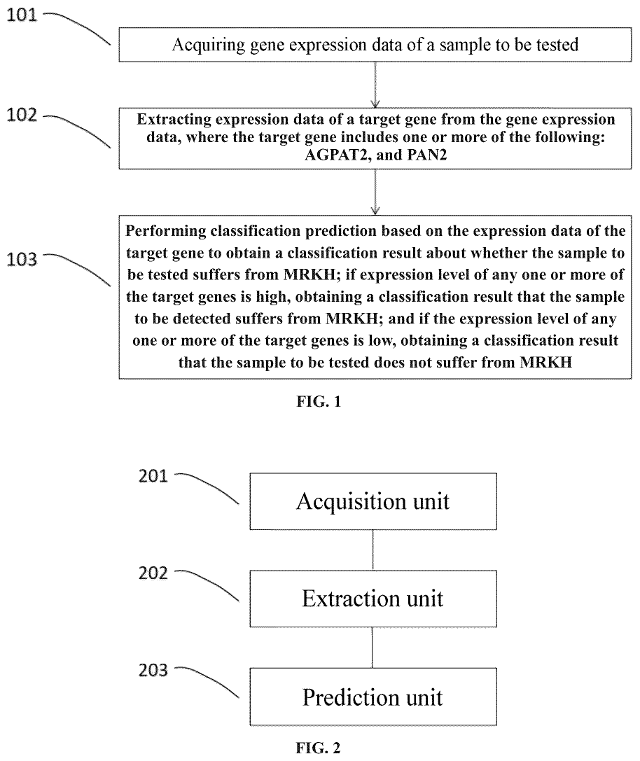

FIG. 1 is a flow diagram of a prediction method for MRKH syndrome according to an embodiment of the present disclosure. Specifically, the method includes the following steps:

101. Acquiring gene expression data of a sample to be tested.

In an embodiment, the gene expression data is exome sequencing or genome sequencing data of peripheral blood DNA (deoxyribonucleic acid).

102. Extracting expression data of the target genes from the gene expression data, where the target genes include one or more of the following: AGPAT2, and PAN2.

In an embodiment, the target gene further includes one or more of the following: PAX8, BMP7, and GREB1L. Further, the target gene further includes any one or more suboptimal genes as follows: MT1F, BMP7, and AGPAT2. Further, the target gene further includes any one or more digenic combinations as follows: EVC2-KANK1, CYP2A7-CPSF3L, NOS1-AICDA, AGPAT2-MAMDC4, BMP7-SH2D6, and PAX8-MYO7A.

103. Predicting by classification based on the expression data of the target genes to obtain a classification result about whether the sample to be tested suffers from MRKH. If an expression level of any one or more of the target genes is high, a classification result that the sample to be tested suffers from MRKH is obtained; and if the expression level of any one or more of the target genes is low, a classification result that the sample to be tested does not suffer from MRKH is obtained.

In an embodiment, the expression levels of any one or more of the target genes at an 8th week and an 11th week of the sample to be tested are acquired. If the expression levels of the target genes in uterine epithelial cells at the 8th week and Wolffian Duct epithelial cells at the 11th week are high, a result that the sample to be tested suffers from MRKH is obtained. The sample to be tested is a developing embryo. Further, based on a result that the embryo to be tested suffers from MRKH, a proposal for further examination is given. If the embryo to be tested suffers from MRKH and is complicated with multisystem malformations (severe cases), a proposal for abortion is given. If the embryo to be tested suffers from MRKH but only has reproductive tract malformation, a protocol of evaluating the function of urogenital system after birth is given. Based on the evaluation results, a surgical protocol is made.

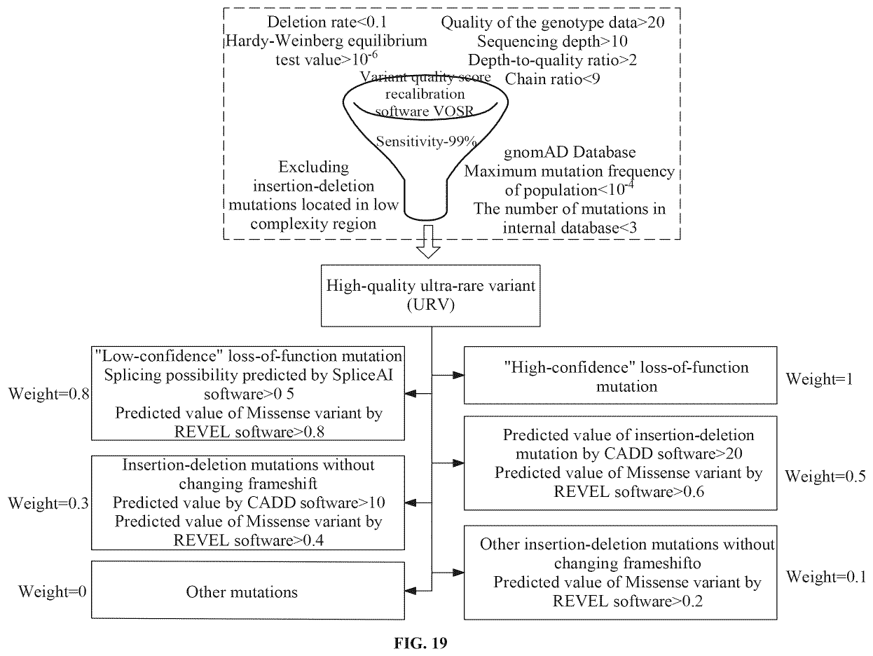

In an embodiment, the classification result is obtained based on a prediction model. A construction method for the prediction model includes the following steps:
acquiring genome sequencing data of a sample from a training set and corresponding clinical characteristics of the sample, where the clinical characteristics are collected from a MRKH patient and an healthy people;
decomposing an ultra-rare variant in the genome sequencing data into gene units by using a weighting system, and performing load testing on the MRKH patient and healthy people to obtain a target gene with significant expression in whole exome and an expression level of the target gene. Alternatively, the weighting system is a six-layer weighting system. Specifically, FIG. 19 is a schematic diagram of specific flow of a six-layer weighting system according to an embodiment of the present disclosure, which includes the following steps:

(1) screening mutations that meet the following requirements: a) a deletion rate is less than 0.1; b) A Hardy Weinberg Equilibrium test value is greater than 10-6; c) genotype quality is greater than 20; d) a sequencing depth is greater than 10; e) a depth-to-quality ratio is greater than 2; f) a chain ratio is less than 9; g) a maximum mutation frequency of gnomAD database population is less than 10-4; h) the number of mutations in an internal database is less than 3; and i) if the mutation is an insertion-deletion mutation (Indel), the mutation located in a low complexity region is excluded.

(2) The mutation obtained from initial screening is further screened by variant quality score recalibration software VQSR with a sensitivity of 99%, thus obtaining a high-quality ultra-rare variant (URV).

(3) The function of mutation is annotated by using software such as LOFTEE, SpliceAI, CADD and REVEL, and the harmfulness of mutation is interpreted according to annotation results, and then the six-layer weighted ranking is carried out in turn.

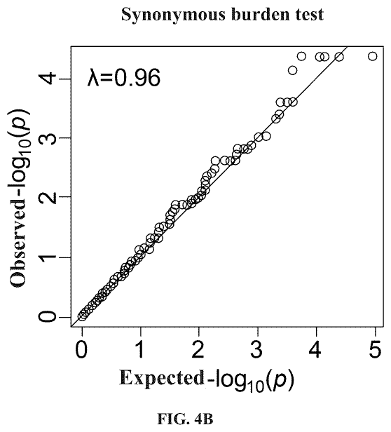

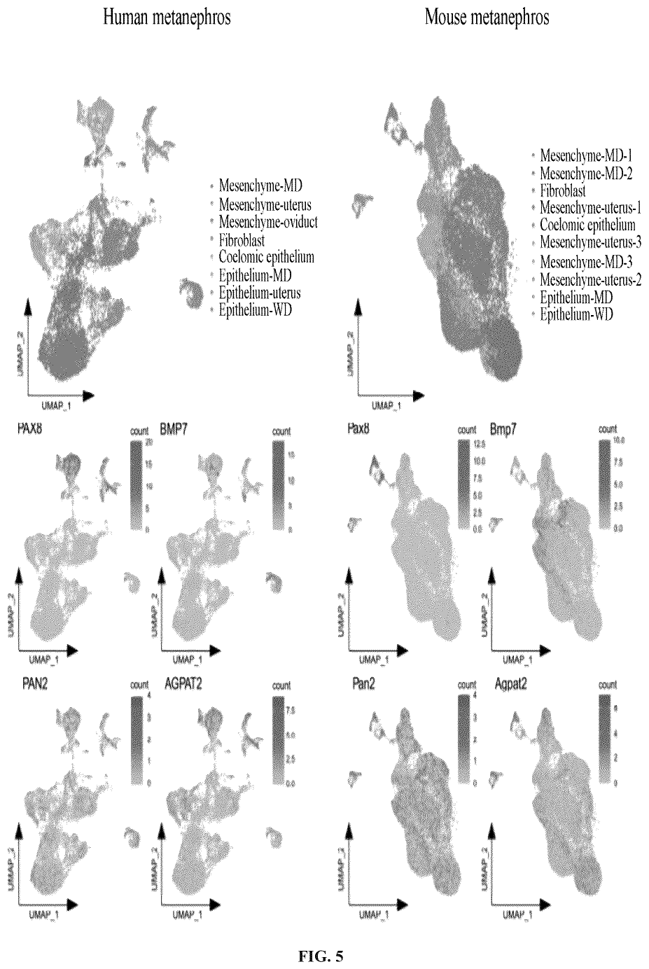

Modeling based on the expression level of the target gene and the clinical characteristics to obtain a prediction model. Specifically, a way to acquire the target gene includes: 727 patients diagnosed with MRKHS were recruited, including 476 patients with solitary MRKHS and 251 patients with various congenital abnormalities. After DNA sequencing, data processing and quality control, ES/GS data of 716 MRKHS probands and 2402 control individuals in an analysis set were reserved. The six-layer weighting system is used to fold the ultra-rare variant (URV, with the largest population allele frequency of less than 0.01%) into gene units, and an ACAT package is used for case-control burden test. The five genes have achieved the significance of the whole exome (false discovery rate [FDR] is less than 0.05, FIG. 4-FIG. 6). PAX8 and BMP7 have been identified in previous candidate gene studies for a subset of the cohort (N=442). In many recent studies, GREB1L has also been reported as the pathogenic gene of MRKHS. PAN2 and AGPAT2 represent new disease genes of MRKHS.

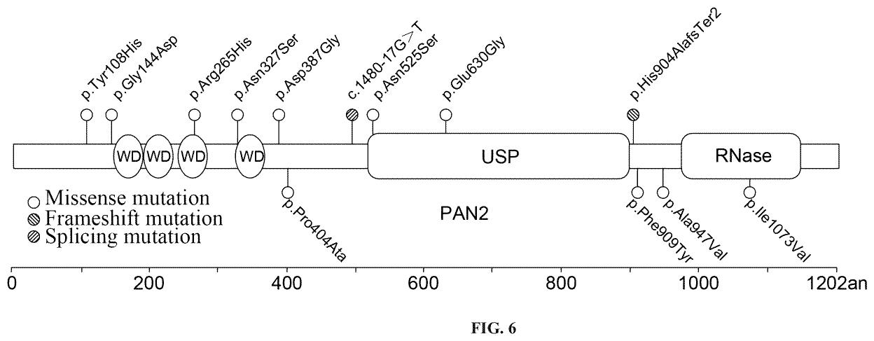

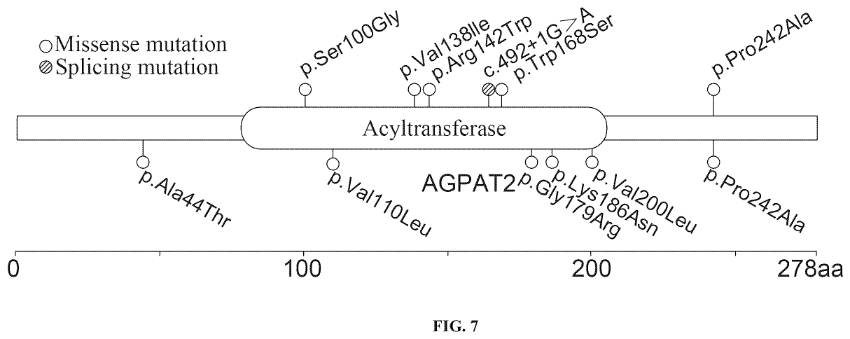

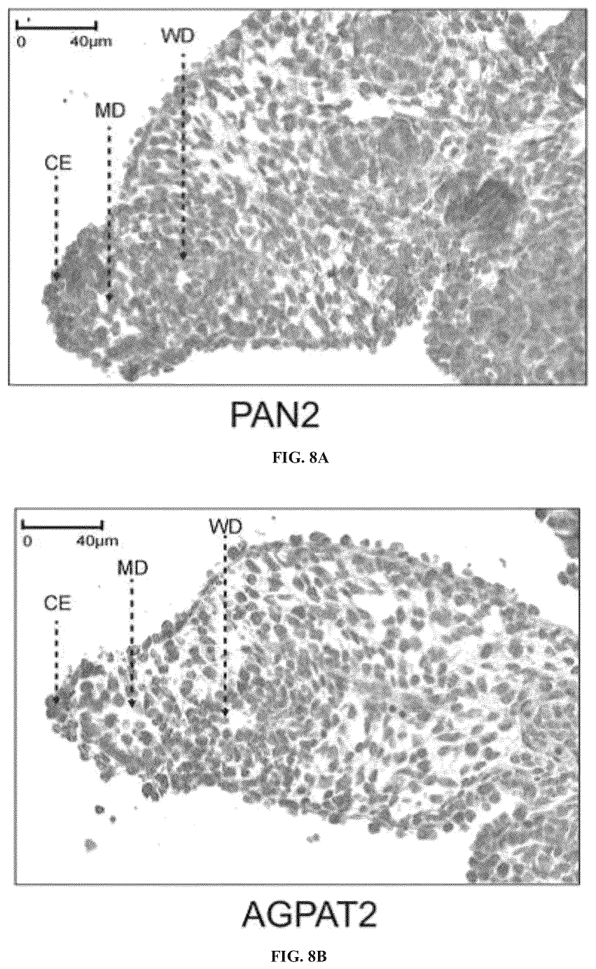

PAN2 encodes a subunit of deadenylation complex, which can regulate mRNA stability and translation efficiency. PAN2/Pan2 is widely expressed in metanephros of human and mouse (FIG. 5 and FIG. 8). Recently, it is reported that there is a biallelic variant of PAN2 in a neurodevelopmental syndrome, which has many congenital abnormalities, including genitourinary system abnormalities, such as renal dysplasia. Nine heterozygous URVs ((FDR=0.015, odds ratio [OR]=18.4) were found in the patient with MRKHS, which are enriched in N-terminal of PAN2 protein (FIG. 6). No second harmful allele was found in the patients, and none of the patients had renal abnormalities, which indicated that there was a complex genetic mechanism, not Mendelian inheritance. AGPAT2 encodes O-acyltransferase and participates in the metabolism of lysophosphatidic acid. AGPAT2 is specifically expressed in MD, WD and CE of human and mouse embryos (FIG. 5 and FIG. 8). The pathogenic variant of AGPAT2 will lead to autosomal recessive congenital lipodystrophy, and it is reported that there will be urogenital phenotypes, such as labial hypertrophy and female fertility decline. Nine URVs were found in the patients (FDR=0.044, OR=12.4, FIG. 7). It should be noted that missense URV (c.298A>Gp.Ser100Gly) recurred in three patients, but not in the control individuals. This variant is in an acyltransferase domain of AGPAT2, and it is predicted that it may destroy a hydrogen bond between Ser100 and Asp103 (a key catalytic residue). No second harmful allele was observed in the patients with AGPAT2 variant, and there was no sign of lipodystrophy.

Alternatively, a construction method for the prediction model further includes the following steps:

acquiring gene expression data of (216) significant genes in metanephric cells of human and mouse at different embryonic stages; extracting gene expression data of the significant genes after removing a whole exome signal of the target gene from the gene expression data of the significant genes to obtain a first suboptimal gene related to MRKH, where the first suboptimal genes include: MT1F; and modeling based on the expression levels of the target gene and the first optimal gene as well as the clinical characteristics to obtain a prediction model.

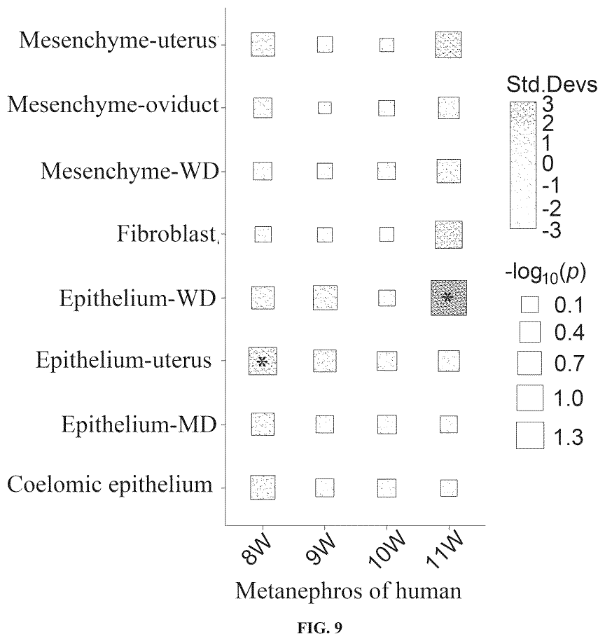

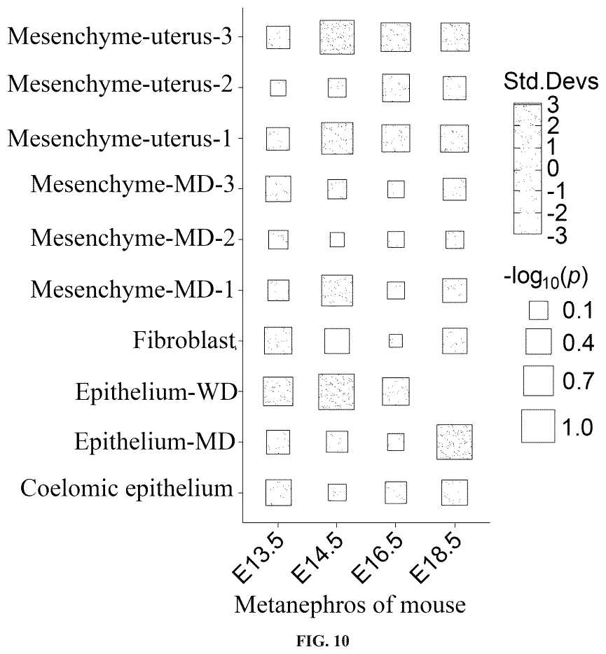

Specifically, an acquisition way of the first suboptimal gene includes the following steps: examining co-expression of 216 nominally significantly expressed genes in the burden test (a P value of less than 0.01 was defined to minimize noise) in uterine and metanephros cell types of human and mouse at different embryonic stages to further understand the role of MRKHS-related genes in uterine development. In the human embryo, the expression of MRKHS-related genes is enriched in the uterine epithelium at an 8th week of pregnancy and the Wolffian Duct (WD) epithelium at an 11th week, which proves the biological correlation among the load signals (FIG. 9). Although five whole exome signals are removed, the enrichment still remains, indicating that the suboptimal burden test gene also leads to disease tendency. In the suboptimal genes, MT1F is preferred, which is specifically expressed in human uterine epithelium and mouse MD/WD epithelium. As a member of metallothionein family, MT1F is also known as the endometrial marker of menstrual cycle of an adult. It should be noted that four URVs in MT1F were found in the case, but not in the control, which indicated that the ability to detect the MTF1 in the whole exome-wide might be lacking. Consistent with the data of the human, the expression of MRKHS-related genes in the metanephros of the mouse is slightly enriched in E14.5 WD epithelium and E18.5 MD epithelium (FIG. 10). Interestingly, WD shows a strong enrichment of MRKHS-related genes in human and mouse, indicating the dependence of MD/uterus development on WD.

As shown in FIG. 9 to FIG. 12, Std dev means a standard deviation deviates from the mean; * indicates that P is less than 0.05, and #, denotes a whole exome significant signal.

In some embodiments, a construction method for the prediction model further includes the following steps: extracting MRKH-related digenic combinations from the genome sequencing data, and screening from the MRKH-related digenic combinations to obtain a first digenic combination, where the first digenic combination includes EVC2-KANK1, CYP2A7-CPSF3L, NOS1-AICDA. Specifically, the mutation burden of digenic combinations in the whole exome-wide is studied by RareComb. A co-expression pattern is analyzed according to scRNA-seq data of the metanephros of the embryo, thus understanding the biological relevance of the digenic combination. Due to the lack of parental samples, there is no systematic in-home isolation analysis of these combinations.

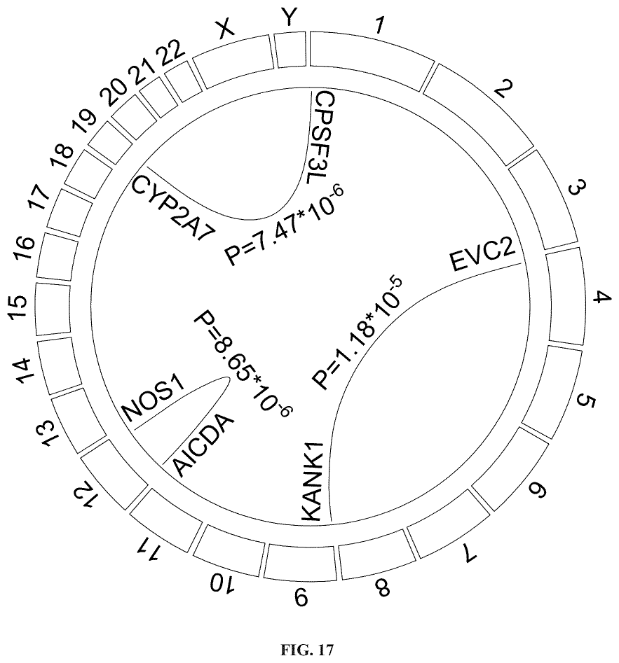

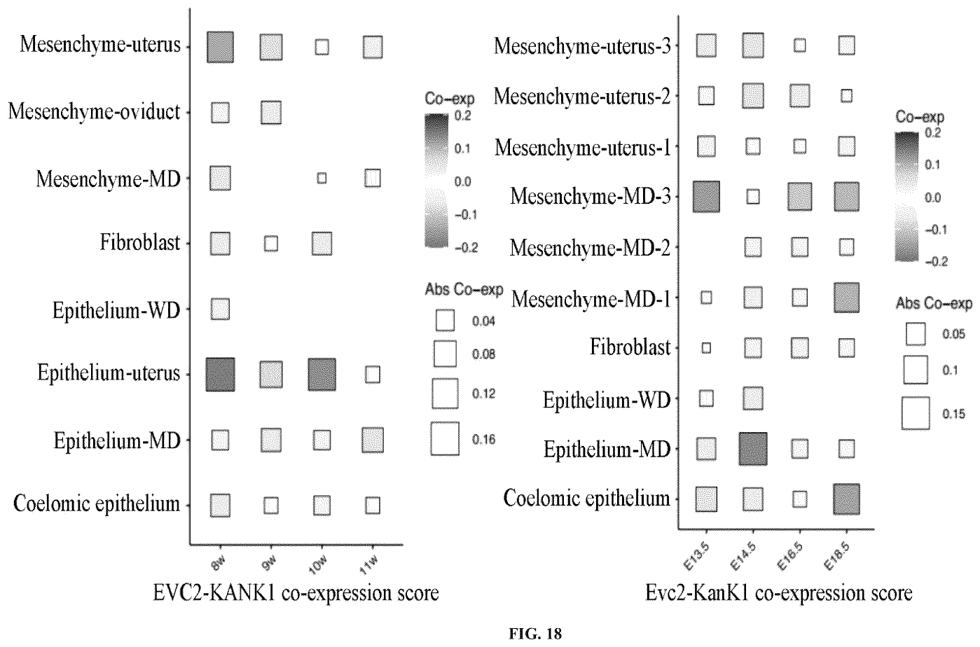

992 MRKHS-related digenic combinations of the patients were identified, including three combinations with whole exome significance (FIG. 17). EVC2-KANK1 is introduced emphatically, which includes three digenic combinations in the patients with MRKHS, but not in the control group. All three patients carry LoF variant of one gene (EVC2 or KANK1) and harmful variant of another gene. KANK1 was specifically expressed in MD, WD and uterine epithelium, and abnormal uterine morphology was reported in Evc2−/−mouse (www.mousephenotype.org). In addition, it was observed that EVC2 and KANK1 were strongly co-expressed in uterus/MD epithelium of human and mouse (FIG. 18), which indicated that EVC2 and KANK1 interacted closely in a cell-type-specific molecular network. According to previous protein-protein interaction (PPI) studies, both EVC2 and KANK1 interact with YWHA protein, which may mediate their co-expression. YWHA represents a highly conserved protein family, and used to regulate important intracellular events, including cell cycle control, signal transduction, and embryonic development.

As shown in FIG. 17 to FIG. 18, Co-exp represents co-expression score, and Abs Co-exp denotes an absolute value of the co-expression score.

A second digenic combination is determined from the digenic combinations with the target gene. The second digenic combination includes: AGPAT2-MAMDC4, BMP7-SH2D6, and PAX8-MYO7A. Specifically, a potential digenic partner of five whole exome burden signals is also studied; a digenic combination including the target gene is the potential digenic partner. Three nominally significant digenic combinations are identified; each combination was observed for three times in the patient cohort with MRKHS, but was not observed in the control cohort. Among them, the AGPAT2-MAMDC4 combination shows strong co-expression in human MD mesenchymal cells and mouse MD epithelial cells. However, the two variants of AGPAT2 and MAMDC4 are linked on chromosome 9, so it is unlikely to conform to the typical digenic inheritance. In addition, BMP7-SH2D6 combination shows moderate co-expression in human MD and uterine epithelium.

It is reported that some MRKHS-related genes exist in dominant Mendelian diseases that affect other organ systems except uterus. For example, haploid deficiency of PAX8 is related to autosomal dominant congenital hypothyroidism. However, in more than 30 cases with PAX8-related hypothyroidism, uterine malformation had not been reported before the last report, indicating that the penetrance of uterine phenotype related to PAX8 (a gene with obvious haploid deficiency) decreased. It is considered that the uterus may be more tolerant to dose loss of PAX8 than the thyroid, this is because the complex developmental regulation and compensation mechanism of the uterus is evolved to protect fertility. Therefore, digenic inheritance represents a reasonable explanation for the “absence of penetrance” of uterine phenotype. Important digenic partner for three of the five whole exome burden signals are determined, and two of them show the co-expression pattern in the metanephros. New gene combinations that are not independently related to MRKHS but are supported by powerful statistical or biological evidence have also been identified. It is considered that the “double hit” by the genes in the same network may have more harmful effects on the molecular regulatory network of the developing uterus. In the present disclosure, the importance of studying different components of a complex genetic structure by using different statistical tools is emphasized.

Modeling based on the expression levels of the target gene and the digenic combination as well as the clinical characteristics to obtain a prediction model.

In some embodiments, a construction method for the prediction model further includes the following steps:

acquiring transcriptome sequencing data of a sample from a training set, and performing hierarchical clustering on gene expression patterns of the transcriptome sequencing data to obtain at least one co-expressed gene cluster; screening from the co-expressed gene cluster to obtain a gene that does not reach the significance of the whole exome but is clustered into a same gene module as the whole exome signal, recording the gene as a second suboptimal gene. The second suboptimal gene includes BMP7, and AGPAT2. Modeling based on the expression levels of the target gene and the suboptimal gene, as well as the clinical characteristics to obtain a prediction model.

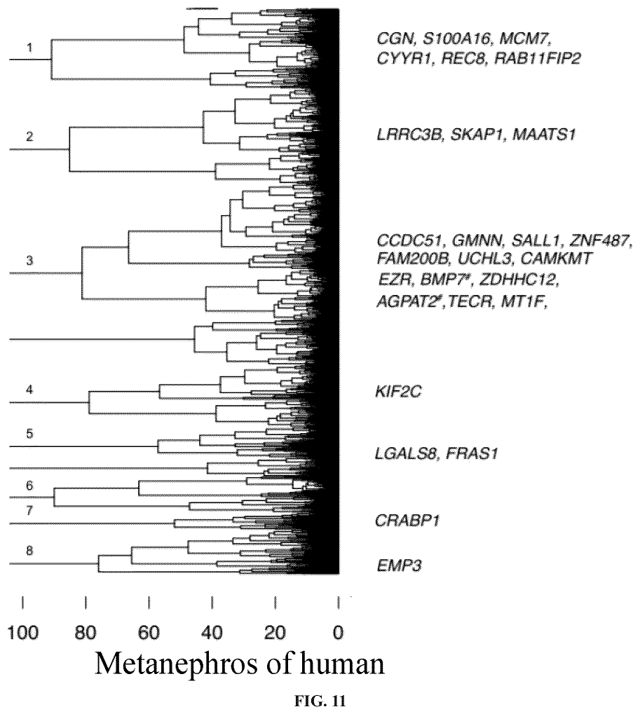

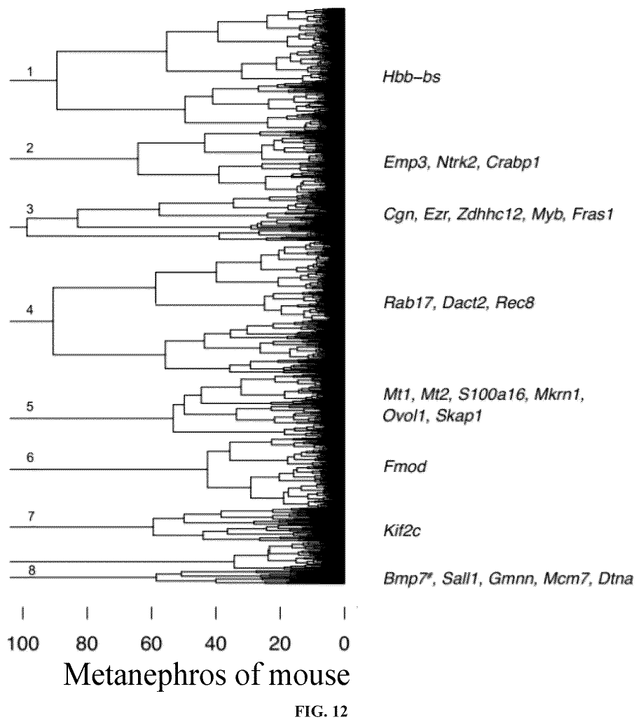

Specifically, an acquisition way of the second suboptimal gene includes the following steps: performing hierarchical clustering according to the co-expression pattern in scRNAseq data. Four co-expressed gene clusters (i.e., gene modules) usually capture key signal pathways and molecular networks. Therefore, priority is given to those genes that do not reach the significance of the whole exome but are clustered in the same gene module as the whole exome signal (FIG. 11 and FIG. 12). A gene module #3 from human embryo data is rich in the burden test signal, including BMP7 and AGPAT2 which are significant in the whole exome (FIG. 11). In other nominally significant genes in this module, the focus is on MT1F, which is also captured and prioritized through above expression analysis. SALL1 regulates BMP7 expression, which is related to human vaginal hypoplasia. The gene module of mouse data shows a cluster pattern in which the burden test signals are more dispersed (FIG. 12), indicating that there are differences between the molecular regulations of uterine development of human and mouse.

Further, a construction method for the prediction model further includes the following steps: decomposing an ultra-rare variant in the co-expressed gene cluster into genes by using a weighting system, summing a maximum weight burden score of each gene after decomposition to calculate a genome mutation burden score, and fitting the genome mutation burden scores to obtain a fitting result; and modeling based on the expression levels of the target gene and the suboptimal gene, as well as the fitting result and the clinical characteristics to obtain a prediction model. Specifically, in order to measure the combined effect of mutations in biologically related gene clusters, the mutation burden of the gene set is analyzed. First, a qualified variant is folded into genes using the same filtering and weighting criteria as in the gene level burden test. The burden scores of all genes in the gene set are summarized into a gene set level burden score, which is then used to fit a logistic regression model to predict a disease state. Firstly, DNA sequencing of a sample cohort is carried out; the gene frequency and mutation function are annotated by a mutation effect prediction tool to screen out the rare variant in each gene. The weight value is endowed to each rare variant according to the harmful degree thereof to the gene function, and the genomic mutation burden score of each gene in a case sample and a control sample are calculated by comprehensive weighting.

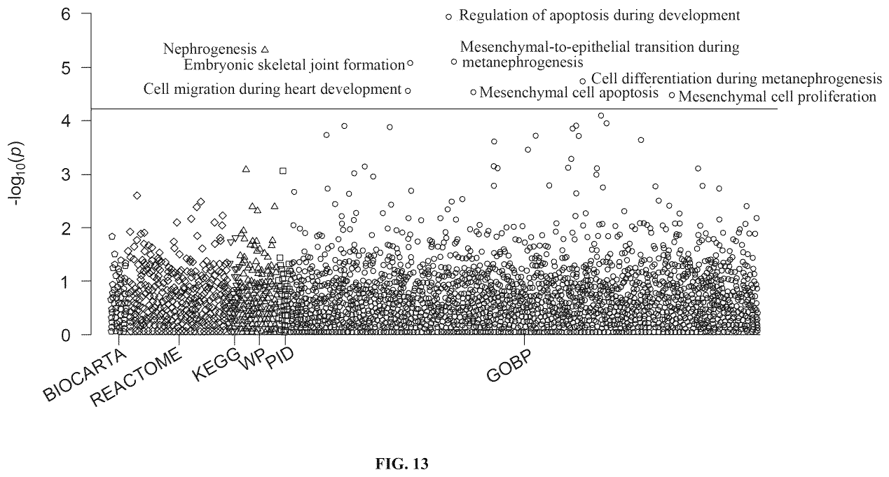

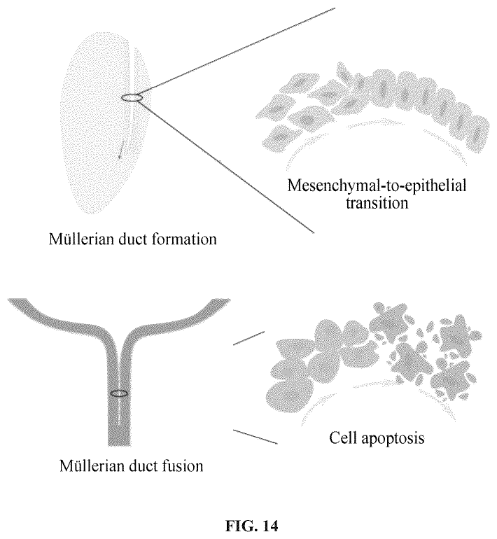

Firstly, the mutation burdens of genomes representing biological processes and molecular pathways in a molecular signatures database (MSigDb) are tested. “Regulation of apoptosis process involved in development” and “Mesenchymal to epithelial transition involved in metanephric morphogenesis” in a gene ontology (GO) database are popular searches (FIG. 13), which are consistent with their important roles in morphogenesis and formation of female reproductive tract (FIG. 14). Genes involved in the development of skeletal joints and heart are also enriched in MRKHS, indicating that there is a shared molecular network among mesoderm-derived organs (FIG. 13). As expected, the “nephrogenesis” pathway from Wiki Pathway also has too much mutation burden in the patient with MRKHS (FIG. 13).

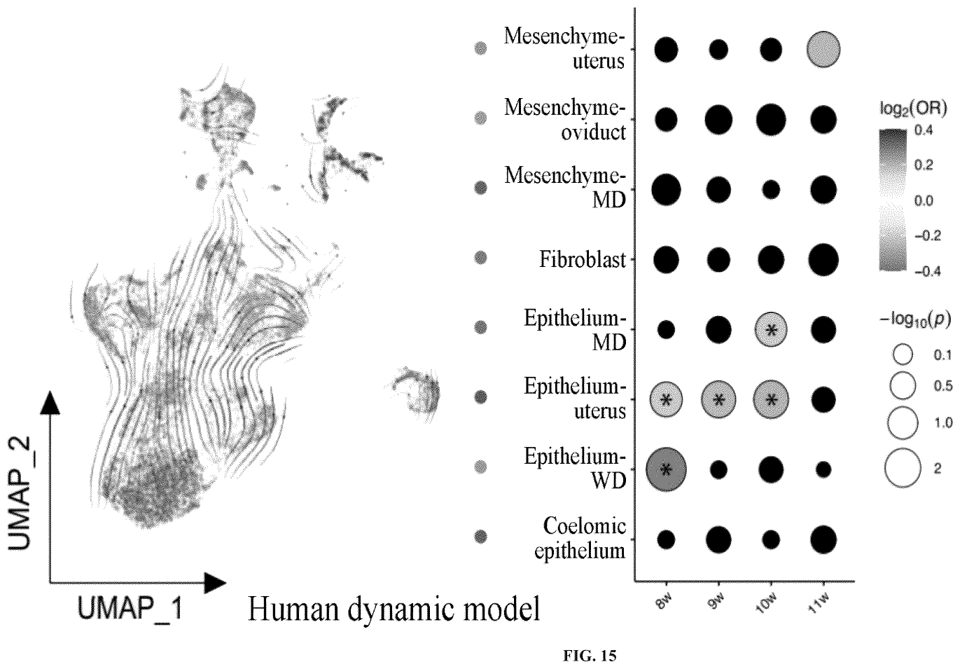

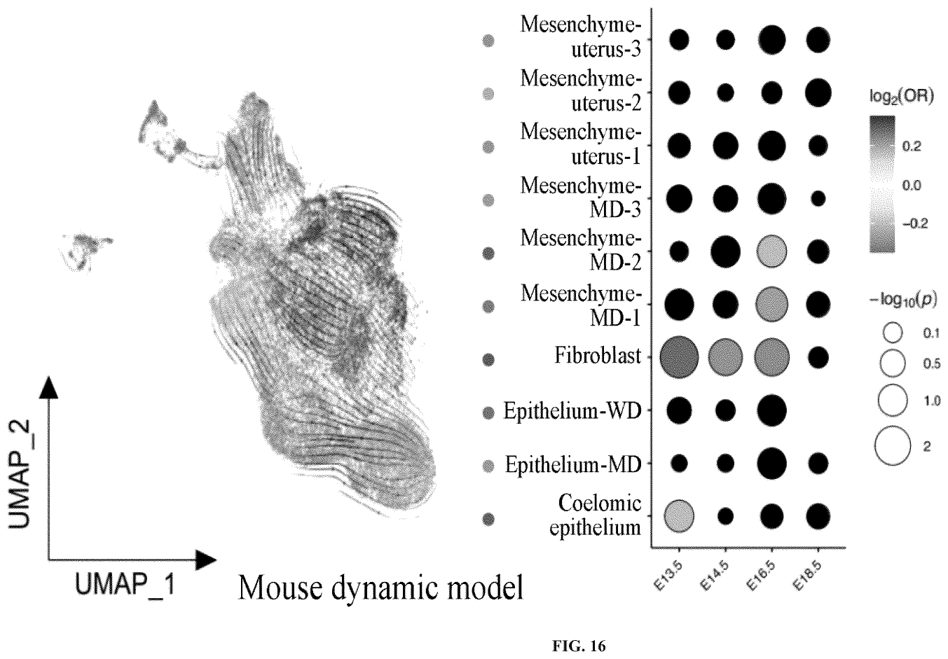

Cell-type-specific driver genes are derived from scRNA-seq speed analysis, which takes a ratio of spliced/unspliced mRNA into consideration, rather than the absolute expression levels of genes. The top 500 driver genes of each cell type were searched, and the burden test at the gene set level was carried out. Different from the expression enrichment analysis that the WD epithelium shows the highest signal, the dynamic driver genes of MD epithelium have the most significant mutation burden in the patients with MRKHS (FIG. 15). No significant signal of the driver gene from the mouse data is observed (FIG. 16), which indicates that there are species differences in the regulatory network of uterine development again.

In an embodiment, the method further includes the following steps: acquiring a genome mutation burden score of the sample to be tested, and predicting a classification result of high or low risk of suffering from MRKH according to the score; if the genome mutation burden score is high, obtaining a classification result that the sample to be tested has a high risk of suffering from MRKH; and if the genome mutation burden score is low, obtaining a classification result that the sample to be tested has low risk of suffering from MRKH.

Further, the genome mutation burden score is obtained through the following ways: screening out rare variants in the target genes by using a mutation effect prediction tool, endowing weight values respectively according to the harmful degree of the rare variants to gene function, and comprehensively weighting to calculate the genome mutation burden score of the sample to be tested.

Alternatively, the genome mutation burden score is further obtained through the following ways: screening out a rare variant in each gene by using the mutation effect prediction tool, endowing weight values respectively according to the harmful degree of each rare variant to the gene function, and comprehensively weighting to calculate the genome mutation burden score of the sample to be tested.

FIG. 2 is a prediction system for MRKH syndrome according to an embodiment of the present disclosure. The system includes:
an acquisition unit 201, configured to acquire gene expression data of a sample to be tested;
an extraction unit 202, configured to extract expression data of a target gene from the gene expression data, wherein the target gene includes one or more of the following: AGPAT2, and PAN2; and
a prediction unit 203, configured to perform classification prediction based on the expression data of the target gene to obtain a classification result about whether the sample to be tested suffers from MRKH, obtain a classification result that the sample to be tested suffers from MRKH if expression level of any one or more of the target genes is high, and obtain a classification result that the sample to be tested does not suffer from MRKH if the expression level of any one or more of the target genes is low.

Alternatively, the system further includes:
a second acquisition unit, configured to acquire a genome mutation burden score of the sample to be tested; and
a second prediction method, configured to predict the classification result of high or low risk of suffering from MRKH according to the score, and obtain a classification result that the sample to be tested has a high risk of suffering from MRKH if the genome mutation burden score is high, and obtain a classification result that the sample to be tested has a low risk of suffering from MRKH if the genome mutation burden score is low.

Alternatively, the system further includes:
a third acquisition unit, configured to acquire the expression levels of any one or more of the target genes at an 8th week and an 11th week of the sample to be tested;
a third prediction method, configured to obtain a result that the sample to be tested suffers from MRKH if the expression levels of the target genes are high in uterine epithelial cells at the 8th week and Wolffian Duct epithelial cells at the 11th week, where the sample to be tested is a developing embryo; and
a protocol recommendation unit, configured to provide a further examination protocol based on the result that the embryo to be tested suffers from MRKH.

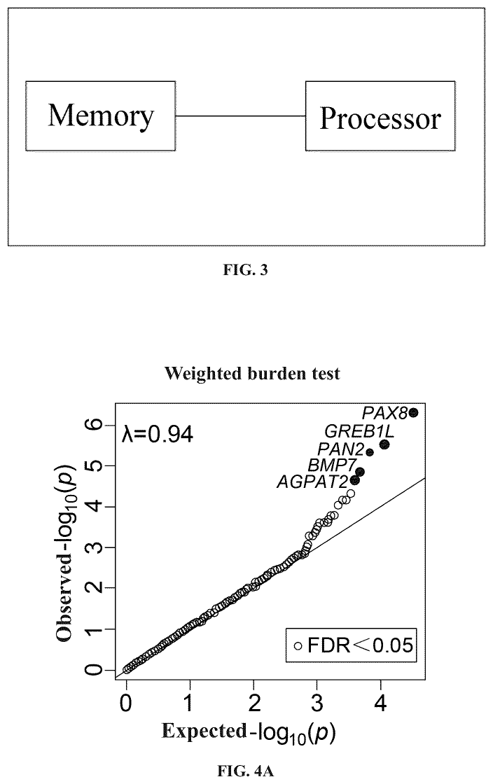

FIG. 3 is a computer device according to an embodiment of the present disclosure. The device includes a memory, and a processor. A program instruction is stored in the memory, and a program instruction is called in the processor, and the program instruction, when being executed, is configured to implement the steps of the method above.

An embodiment of the present disclosure further provides a computer readable storage medium, on which a computer program is stored. The computer program, when being executed by a processor, is configured to implement the steps of the method above.

An embodiment of the present disclosure further provides a computer program product, including a computer program. The computer program, when being executed by a processor, is configured to implement the steps of the method above.

In an embodiment, the sample from the training set is as follows: under the framework of DISCO (Deciphering Disorders Involving Scoliosis and COmorbidities), 727 probands (families) diagnosed with MRKHS in Peking Union Medical College Hospital (Beijing, China) and Shenzhen Luohu Hospital (Shenzhen, China) were recruited from November 2012 to Nov. 7, 2020. (Study of complications). All probands received karyotype analysis, female sex hormone test, ultrasound examination of urinary system, X-raying of spinal cord, and electrocardiogram examination. About half of the patients underwent pelvic magnetic resonance imaging. The diagnostic criteria of MRKHS are as follows: (1) uterine hypoplasia by pelvic ultrasound examination or pelvic MRI, and (2) normal female karyotype (46, XX). The control individuals were from female participants received exome sequencing in the Medical Research Center of Peking Union Medical College Hospital for clinical or research purposes. Individuals recorded with urogenital malformation or other congenital abnormalities were excluded. The retained control data set includes 2504 unrelated female individuals. This study was approved by the Ethics Committee of Peking Union Medical College Hospital (ZS-1858).

DNA sequencing: peripheral blood DNA was subjected to exome sequencing(ES) or genome sequencing (GS). The exome sequencing of 442 probands (families) has been reported in detail before (McLaren W, Gil L, Hunt S E, et al. The Ensembl Variant Effect Predictor [J]. Genome Biol, 2016, 17 (1): 122). In this study, four other probands received ES treatment according to the same protocol as previously reported (Chen N, Zhao S, JollyA, et al. Perturbations of genes essential for Müllerian Duct and Wolffian Duct development in Mayer-Rokitansky-Kuster-Hauser syndrome [J]. Am J HumGenet, 2021, 108 (2): 337-45). An average depth of ES is 70×. In addition, 281 probands (families) received GS with an average depth of 38×. For GS, according to the optimized protocol of manufacturers, a PCR-free sequencing library is prepared using KAPA Hyper Prep kit (KAPA Biosystems, Kusatsu, Japan). Multiplex sequencing is performed using Illumina (Illumina, San Diego, CA, USA) NovaSeq platform. The sequencing method for control samples has been described in previous studies.

The raw data from ES and GS were processed by the Peking Union Medical College Hospital Pipeline (PUMP). Sentieon software is used to generate a gvcf document for each sequencing data and to perform across-sample joint calling for a series of variant level quality control (QC). After performing the variant level QC, the population stratification is tested by principal component analysis (PCA). PCA is performed using PLINK (version 1.90) on the basis of combined genotypes of internal subjects and 1000 genome phase III population. Then, sample-level quality control is carried out to remove individuals with low sequencing quality, or with inter-sample relevance or genetic inference as men.

Burden analysis of rare variants in gene pool: the gene-based burden analysis of ultra-rare variants (URV) is restricted, and these variants are defined as those with the maximum allele frequency (0.01%) and allele counts (3 in case-control cohort) of gnomAD population. cDNA and protein level consequences of URVs are annotated using ENSEMBL variant effect predictor (VEP, version 103). Only variants containing annotations in canonical transcript of ENSEMBL are analyzed. Each variant is assigned a weight score according to its variant type and predicted harmfulness. ACAT package is used for weighted burden test of each gene. Multiple tests are adjusted using a false positive rate (FDR) method.

Associated analysis of gene set level: curated gene sets (gene number (10)) representing biological processes and pathways are retrieved from the molecular signatures database (MSigDB). Cell type-specific driver genes are derived from scRNA transcriptome data (described in the following section). The pretreatment and weighting of URV are the same as those in gene level burden analysis. The mutation burden score of a given genome is calculated by summing the maximum weighted burden score of each gene in the genome. The genome level burden score is used to fit a logistic regression model to predict the disease state. The FDR (false discovery rate) method is used to adjust the multiple tests.

### Digenic Combination Analysis:

RareComb package is used to identify MRKHS-related digenic combinations with rare variants (minor allele frequency, MAF (1%)) in different genes. Only the predicted harmful variant (weight (0.5)) is included to reduce the influence of background noise. Due to limited sample size, only digenic combination, but not oligogene combination, is analyzed. The co-expression matrix from single cell transcriptome data (which will be described in the following section) is used to study the biological relationship between the digenic combinations. For each candidate digenic pair, the genes with co-expression scores of metanephric cell types of human and mouse at different time points are examined. The distribution of the co-expression scores of all gene pairs in the co-expression matrix of all significant digenic combinations is also tested. An absolute value of the co-expression score is obtained by comparing the digenic combination and a random pair by using two-sample student t test.

Anatomy of mesonephros in human and mouse: According to the protocol (JS-3482) approved by the Ethics Committee of Peking Union Medical College Hospital, human fetuses at the 8th week, 9th week, 10th week and 11th week are obtained by selective pregnancy termination. Mouse embryos of E12.5, E14.5, E16.5 and E18.5 are collected from C57BL/6 wild mouse. The mesonephric tissues are dissected from human or mouse embryos under microscope. In addition, the mesonephros of E13.5 mouse is collected for immunohistochemical staining of candidate genes.

Sequencing and analysis of single cell transcriptome: Mesonephric tissues from human (w8, w9, w10 and w11) and mouse embryos (E12.5, E14.5, E16.5 and E18.5) are digested and processed to produce cell suspension. According to the instructions of the manufacturer (10× Genomics, Pleasanton, CA), a droplet-based library is prepared using the 10× Genomics Single Cell 3prime kit v3. Sequencing is carried out on Illumina NovaSeq 6000 platform. CellRanger and Seurat are used to preprocess the scRNAseq data. Human or mouse embryos at different time points are pooled together, and subjected to cell clustering using the Louvain algorithm. According to the known genetic markers of developing uterus and other tissues, cell clusters are manually annotated.

A generalized dynamics model of sc Velo software is used to analyze RNA velocity based on splicing dynamics. Cell-type-specific driver genes are defined by genes with high probability in a dynamic model. The top 500 driver genes of each cell type are searched and subjected to gene set burden test.

COTAN is used to calculate the co-expression score between each gene pair according to the scRNAseq data. Co-expression matrixs of cell type level and embryo level are generated for downstream analysis. For candidate digenic pairs, the co-expression patterns at the cell type level are examined. For the overall analysis of all digenic pairs, the distribution of the co-expression scores at the embryo level is tested.

An EWCE software is used to enrich MRKHS related genes in scRNA-seq data and analyze. All nominally significant genes from the burden test are included in the test. n=1000 guiding programs are used to test gene expression enrichment across cell types for random background genes.

### Definition

Whole exome sequencing (WES) is a technical means mainly for identifying and studying genetic mutations related to coding regions and Untranslated Regions (UTR) associated with diseases and population evolution through enriching DNA sequence of exon regions by probe hybridization, followed by high-throughput sequencing. Combined with exome data provided by a large number of public databases, it is conducive to better explanation of the correlation between variants and the pathogenic mechanism of diseases. Exon is a protein coding region of human genome, and its DNA can be captured and enriched by a sequence capture technology. Although the exon region only accounts for about 1% of the whole genome, it contains 85% of pathogenic mutations. Compared with the whole genome sequencing, whole exome sequencing is more economical and efficient. Exome sequencing is mainly used to identify and study the variants in the coding region and UTR region associated with diseases and population evolution. Combined with the exon data provided by a large number of public databases, it is conducive to better explanation of the relationship between the obtained variants and diseases.

Tumor mutation burden (TMB) is defined as the total number of substitution and insertion/deletion mutations per Megabase in an exon coding region of the evaluated gene in a tumor sample. TMB reflects the number of gene mutations in tumor tissues. It is generally believed that the more gene mutations in tumor cells, the higher TMB, the more likely it is to produce abnormal protein, and the more likely it is to be identified by the immune system, thus activating the immune response of the human body and benefiting the patient from immunotherapy. It is generally considered that TMB is high when exceeding 20 mutations/Mb (Mb stands for a million bases); and TMB is low when less than 10 mutations/Mb.

Verification results of this verification embodiment show that assigning inherent weights to indications can moderately improve the performance of this method compared with the default settings.

It can be clearly understood by those skilled in the art that for the convenience and conciseness of description, the specific working process of the system, apparatus and unit described above can refer to the corresponding process in the aforementioned method embodiment, and thus will not be repeated here.

In some embodiments provided by the present disclosure, it should be understood that the system, apparatus and method disclosed here may be implemented through other ways. The apparatus embodiments described above are only schematic. For example, the division of the units is only a logical function division, and there may be another division method in actual implementation. For example, multiple units or components may be combined or integrated into another system, or some features may be ignored or not implemented. On the other hand, the mutual coupling or direct coupling or communication connection shown or discussed may be indirect coupling or communication connection through some interfaces, apparatuses or units, which may be in electrical, mechanical or other forms.

The units described as separate components may or may not be physically separated, and the components displayed as a unit may or may not be physical units, that is, they may be in one place, or distributed to multiple network units. A part or all of the units can be selected according to actual needs to implement the purpose of this embodiment.

In addition, various functional units in each embodiment of the present disclosure may be integrated into one processing unit, or they may exist physically alone, or two or more units may be integrated into one unit. The above integrated units can be implemented in the form of hardware, or software functional units.

Those skilled in the art can understand that all or part of the steps in various methods of the above embodiments can be completed by instructing related hardware through programs, and the program can be stored in a computer-readable storage medium, and the storage medium may be a read only memory (ROM), a random access memory (RAM), a magnetic disk or optical disk, etc.

Those skilled in the art can understand that all or part of the steps in the method for achieving the above embodiments can be completed by instructing related hardware through programs. The program can be stored in a computer-readable storage medium, and the above storage medium may be a read-only memory, a magnetic disk, or an optical disk, etc.

The computer device provided by the present disclosure has been described in detail above. For those of ordinary skill in the art, there will be some changes in the specific implementation and application scope according to the concept of the embodiment of the present disclosure. To sum up, the contents of this specification should not be construed as limiting the present disclosure.

What is claimed is:

1. A prediction method for MRKH syndrome, comprising the following steps:
acquiring a gene expression data of a sample to be tested;
extracting expression data of a target gene from the gene expression data, wherein the target gene comprises one or more of the following: AGPAT2, and PAN2;
performing classification prediction based on the expression data of the target gene to obtain a classification result about whether the sample to be tested suffers from MRKH; if expression level of any one or more of the target genes is high, obtaining a classification result that the sample to be tested suffers from MRKH; and if the expression level of any one or more of the target genes is low, obtaining a classification result that the sample to be tested does not suffer from MRKH;
the classification result is obtained based on a prediction model; a construction method for the prediction model comprises the following steps: acquiring genome sequencing data of a sample from a training set and corresponding clinical characteristics of the sample, wherein the clinical characteristics are collected from a MRKH patient and an healthy people; decomposing an ultra-rare variant in the genome sequencing data into gene units by using a weighting system, and performing burden test on the MRKH patient and healthy people to obtain a target gene expressed significantly in whole exome and an expression level of the target gene; and modeling based on the expression level of the target gene as well as the clinical characteristics to obtain a prediction model.

2. The prediction method for MRKH syndrome according to claim 1, wherein the target gene further comprises one or more of the following: PAX8, BMP7, and GREB1L.

3. The prediction method for MRKH syndrome according to claim 1, wherein the target gene further comprises any one or more of the following suboptimal genes: MT1F, BMP7, and AGPAT2.

4. The prediction method for MRKH syndrome according to claim 1, wherein the target gene further comprises one or more of the following digenic combinations: EVC2-KANK1, CYP2A7-CPSF3L, NOS1-AICDA, AGPAT2-MAMDC4, BMP7-SH2D6, and PAX8-MYO7A.

5. The prediction method for MRKH syndrome according to claim 1, wherein the method further comprises the following steps: acquiring a genome mutation burden score of the sample to be tested, and predicting the classification result of high or low risk of suffering from MRKH according to the score; if the genome mutation burden score is high, obtaining a classification result that the sample to be tested has a high risk of suffering from MRKH; and if the genome mutation burden score is low, obtaining a classification result that the sample to be tested has low risk of suffering from MRKH; and
the genome mutation burden score is obtained through the following ways: screening out rare variants in the target genes by using a mutation effect prediction tool, endowing weight values respectively according to harmful degrees of the rare variants to gene function, and comprehensively weighting to calculate the genome mutation burden score of the sample to be tested; or
the genome mutation burden score is further obtained through the following ways: screening out a rare variant in each gene by using the mutation effect prediction tool, endowing weight values respectively according to the harmful degree of each rare variant to the gene function, and comprehensively weighting to calculate the genome mutation burden score of the sample to be tested.

6. The prediction method for MRKH syndrome according to claim 1, wherein the expression levels of any one or more of the target genes at an 8th week and an 11th week of the sample to be tested are acquired, if the expression levels of the target genes are high in uterine epithelial cells at the 8th week and Wolffian Duct epithelial cells at the 11th week, a result that the sample to be tested suffers from MRKH is obtained; and the sample to be tested is a developing embryo.

7. The prediction method for MRKH syndrome according to claim 1, wherein
the construction method for the prediction model further comprises the following steps:
acquiring gene expression data of significant genes in metanephric cells of human and mouse at different embryonic stages;
extracting gene expression data of the significant gene after removing a whole exome signal of the target gene from the gene expression data of the significant gene to obtain a first suboptimal gene related to MRKH, wherein the first suboptimal gene comprises: MT1F;
modeling based on expression levels of the target gene and the first suboptimal gene as well as the clinical characteristics to obtain a prediction model;
the construction method for the prediction model further comprises the following steps:
extracting MRKH-related digenic combinations from the genome sequencing data, and screening from the MRKH-related digenic combinations to obtain a first digenic combination;
determining a second digenic combination from the digenic combinations with the target gene;
modeling based on expression levels of the target gene and the digenic combination as well as the clinical characteristics to obtain a prediction model;
the construction method for the prediction model further comprises the following steps:
acquiring transcriptome sequencing data of a sample from a training set, and performing hierarchical clustering on gene expression patterns of the transcriptome sequencing data to obtain at least one co-expressed gene cluster; screening from the co-expressed gene cluster to obtain a gene that does not reach the significance of the whole exome but is clustered into a same gene module as the whole exome signal, recording the gene as a second suboptimal gene, wherein the second suboptimal gene comprises BMP7, and AGPAT2;
decomposing an ultra-rare variant in the co-expressed gene cluster into genes by using a weighting system, summing a maximum weight burden score of each gene after decomposition to calculate a genome mutation burden score, and fitting the genome mutation burden scores to obtain a fitting result; and
modeling based on expression levels of the target gene and the suboptimal gene, as well as the fitting result and the clinical characteristics to obtain a prediction model.

8. A prediction system for MRKH syndrome, comprising:
an acquisition unit, configured to acquire gene expression data of a sample to be tested;
an extraction unit, configured to extract expression data of target genes from the gene expression data, wherein the target genes comprise one or more of the following: AGPAT2, and PAN2; and
a prediction unit, configured to perform classification prediction based on the expression data of the target genes to obtain a classification result about whether the sample to be tested suffers from MRKH, obtain a classification result that the sample to be detected suffers from MRKH if expression level of any one or more of the target genes is high, and obtain a classification result that the sample to be tested does not suffer from MRKH if the expression level of any one or more of the target genes is low;
the classification result is obtained based on a prediction model; a construction method for the prediction model comprises the following steps: acquiring genome sequencing data of a sample from a training set and corresponding clinical characteristics of the sample, wherein the clinical characteristics are collected from a MRKH patient and an healthy people; decomposing an ultra-rare variant in the genome sequencing data into gene units by using a weighting system, and performing burden test on the MRKH patient and healthy people to obtain a target gene with significant expression in whole exome and an expression level of the target gene; and modeling based on the expression level of the target gene as well as the clinical characteristics to obtain a prediction model.

9. A computer device, comprising a memory, and a processor, wherein a program instruction is stored in the memory, the processor is configured to call the program instruction, and the program instruction, when executed, is configured to execute the steps of the method according to claim 1.

10. A computer readable storage medium, wherein a computer program is stored on the computer readable storage medium, and the computer program, when executed by a processor, is configured to implement the steps of the method according to claim 1.
